(19) United States
(12) Patent Application Publication
(10) Pub. No.: US 2026/0058382 A1
(43) Pub. Date: Feb. 26, 2026

(54) CABLE CONNECTION STRUCTURE

(71) Applicant: Proterial, Ltd., Tokyo (JP)

(72) Inventors: Takumi KOBAYASHI, Tokyo (JP); Takeshi SAWADA, Tokyo (JP)

(73) Assignee: Proterial, Ltd., Tokyo (JP)

(21) Appl. No.: 19/307,800

(22) Filed: Aug. 22, 2025

(30) Foreign Application Priority Data
Aug. 23, 2024 (JP) 2024-143284

(51) Int. Cl. H01R 11/01; A61B 1/00; A61B 1/04; H01B 11/18; H01B 11/20

(52) U.S. Cl. CPC H01R 11/01; A61B 1/00114; A61B 1/042; H01B 11/1869; H01B 11/1895; H01B 11/20

(57) ABSTRACT

A cable connection structure includes an electronic component comprising a plurality of electrodes, a cable including a plurality of electric wires, and a jacket that covers the plurality of electric wires while exposing tip portions of the plurality of electric wires on an electronic component-side, a first covering portion that covers a plurality of connecting portions respectively electrically connecting the plurality of electrodes to the plurality of electric wires, and a second covering portion that covers the first covering portion and the tip portions, wherein the second covering portion has a lower elastic modulus than the first covering portion.

### CROSS-REFERENCE TO RELATED APPLICATIONS

The present application is based on Japanese patent application No. 2024-143284 filed on Aug. 23, 2024, the entire contents of which are incorporated herein by reference.

### TECHNICAL FIELD

The present invention relates to a cable connection structure.

### BACKGROUND OF THE INVENTION

Patent Literature 1 discloses an imaging unit that includes a solid-state imager, a coaxial cable, and a protective resin portion. The solid-state imager has plural electrodes. The coaxial cable has plural electric wires respectively electrically connected to the plural electrodes, and an outer insulating layer that covers the plural electric wires while exposing their end portions on the solid-state imager side. The protective resin portion fills the space between the solid-state imager and the outer insulating layer.
Citation List Patent Literature 1: JP2019-180603A

### SUMMARY OF THE INVENTION

In the imaging unit described in Patent Literature 1, however, flexibility may decrease in the portion with the protective resin portion. Meanwhile, if the protective resin portion is made of a relatively flexible material, connecting portions respectively between plural electric wires and the plural electrodes may not be sufficiently protected.

The invention was made in view of such circumstances and it is an object of the invention to provide a cable connection structure that can reinforce connecting portions respectively between plural electric wires and plural electrodes of an electronic component and also have improved flexibility.

To achieve the above object, the invention provides a cable connection structure, comprising;
an electronic component comprising a plurality of electrodes;
a cable comprising a plurality of electric wires and a jacket that covers the plurality of electric wires while exposing tip portions of the plurality of electric wires on the electronic component side;
a first covering portion that covers a plurality of connecting portions respectively electrically connecting the plurality of electrodes to the plurality of electric wires; and
a second covering portion that covers the first covering portion and the tip portions, wherein the second covering portion has a lower elastic modulus than the first covering portion.

### Advantageous Effects of the Invention

According to the invention, it is possible to provide a cable connection structure that can reinforce connecting portions respectively between plural electric wires and plural electrodes of an electronic component and also have improved flexibility.

### BRIEF DESCRIPTION OF DRAWINGS

FIG. 1 is a schematic configuration diagram illustrating an endoscopic system that includes a cable connection structure in an embodiment of the present invention.

FIG. 2 is an end view showing an end surface of a camera head in the embodiment.

FIG. 3 is a cross-sectional view taken along line III-III in FIG. 2 as viewed in a direction of arrow.

FIG. 4 is a cross-sectional view taken along line IV-IV in FIG. 3 as viewed in a direction of arrow.

FIG. 5 is a perspective view showing an imaging device and a cable in the embodiment.

FIG. 6 is a cross-sectional view showing the cable in the embodiment.

FIG. 7 is a plan view showing an example of a bent state of the cable connection structure in the embodiment.

FIG. 8 is a cross-sectional view showing the imaging device and the cable in a state before being connected in the embodiment.

FIG. 9 is a cross-sectional view showing the imaging device and the cable in a connected state in the embodiment.

FIG. 10 is a cross-sectional view showing a state in which a first covering portion is formed in the embodiment.

### DETAILED DESCRIPTION OF THE INVENTION

### Embodiment

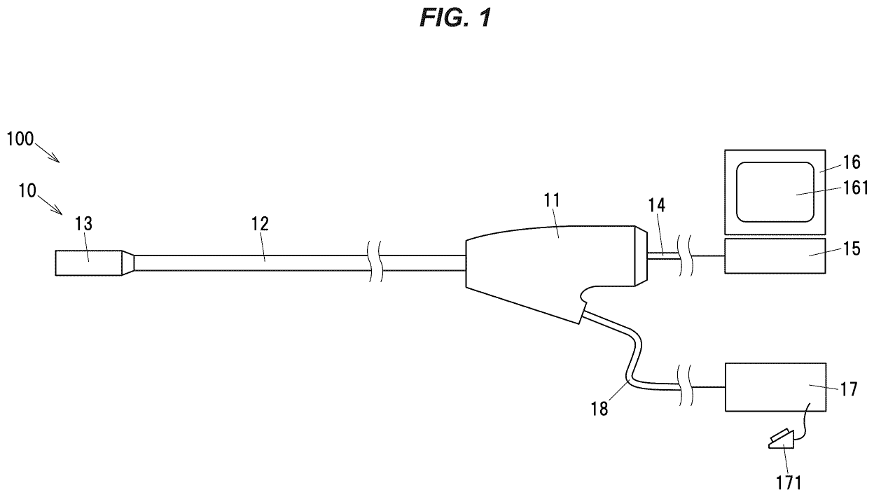

An embodiment of the invention will be described with reference to FIGS. 1 to 10. The embodiment below is described as a preferred example for implementing the invention. Although some part of the embodiment specifically illustrates various technically preferable matters, the technical scope of the invention is not limited to such specific aspects.

FIG. 1 is a schematic configuration diagram illustrating an endoscopic system 100 that includes a cable connection structure 1 in the present embodiment. The endoscopic system 100 includes an endoscope 10, an image processor 15 that processes image information obtained by the endoscope 10, a display device 16 that displays the image processed by the image processor 15 on a screen 161, and a liquid supply device 17 that discharges a camera lens cleaning liquid, etc., in response to operation on a foot switch 171.

The endoscope 10 includes an operating unit 11, an insertion tube 12, and a camera head 13. The operating unit 11 is the part operated by a physician. The operating unit 11 is connected to the image processor 15 through a communication cable 14 and is also connected to the liquid supply device 17 through a hose 18. The insertion tube 12 connects the operating unit 11 to the camera head 13. The length of the insertion tube 12 is, e.g., not less than 1 m and not more than 4 m. The camera head 13, together with a portion of the insertion tube 12, is inserted into the subject's body.

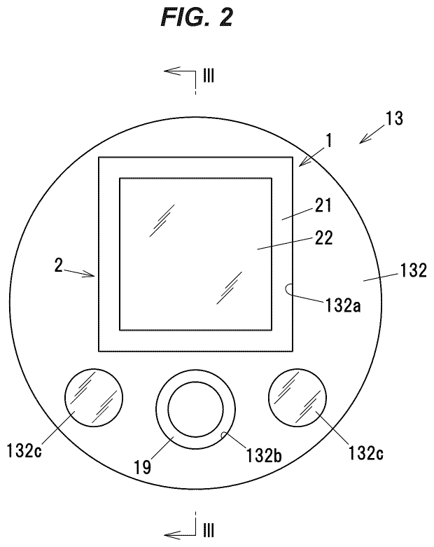

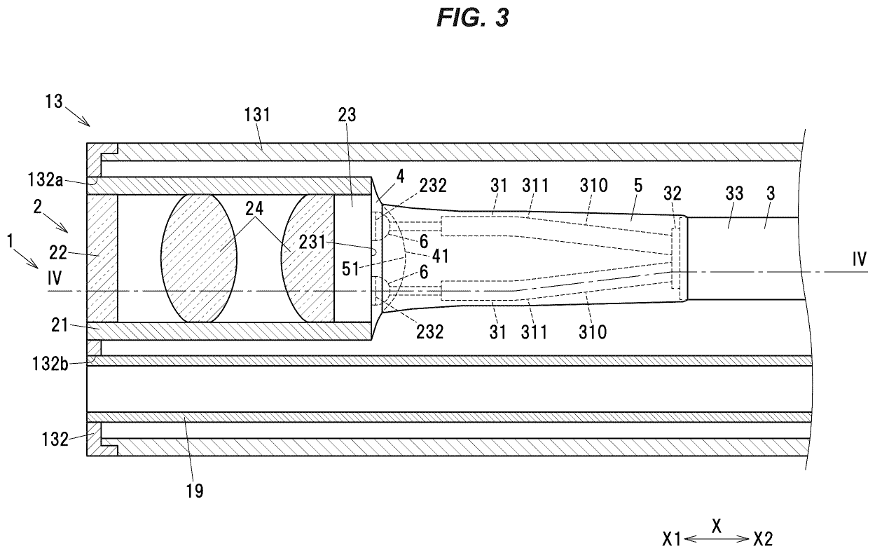

FIG. 2 is an end view showing an end surface of the camera head 13. FIG. 3 is a cross-sectional view taken along line III-III in FIG. 2 as viewed in a direction of arrow. The camera head 13 has an outer cylinder body 131 made of a resin, and a cover body 132 that closes an end of the outer cylinder body 131. A tube 19 to pass a liquid etc. supplied from a liquid supply device 17, an optical fiber (not shown), and the cable connection structure 1 (described later) are inserted inside the outer cylinder body 131 and the insertion tube 12.

The endoscope 10 further has plural steering wires (not shown) which are inserted inside the insertion tube 12 and whose tips are connected to the outer cylinder body 131. For example, two steering wires are connected respectively at two locations spaced 180° apart from each other in the circumferential direction of the outer cylinder body 131, and tension can be applied individually to these two steering wires by operating the operating unit 11. By applying tension to the steering wires, it is possible to bend the tip portion of the camera head 13 in a desired direction and thereby change the imaging direction. In the present embodiment, the camera head 13 is configured to be bendable at a location with a second covering portion 5 (described later) by operating the steering wires. The following is the description of the state in which the tip of the endoscope 10 is not bent, unless otherwise specified.

The cover body 132 has a fitting hole 132a fitted with a tip of an imaging device 2 (described later) of the cable connection structure 1, and a fitting hole 132b fitted with a tip of the tube 19. The cover body 132 also has illumination windows 132c to emit illumination light to illuminate an imaging target area. Light guided by an optical fiber (not shown) is emitted through the illumination windows 132c.

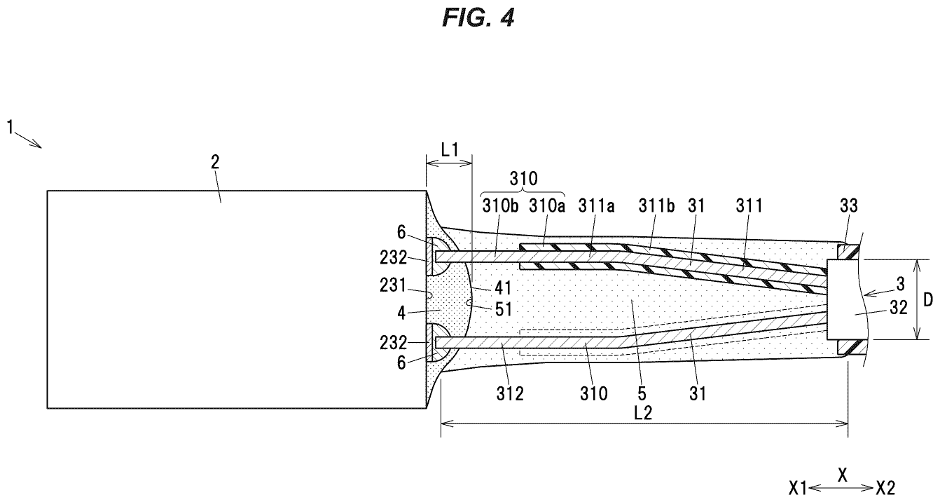

FIG. 4 is a cross-sectional view taken along line IV-IV in FIG. 3 as viewed in a direction of arrow. The cable connection structure 1 includes the imaging device 2, a cable 3, a first covering portion 4, and the second covering portion 5. The imaging device 2 is an electronic component having plural electrodes 232. The cable 3 includes plural electric wires 31, and a jacket 33 that covers the plural electric wires 31 while exposing tip portions 310 of the plural electric wires 31 on the imaging device 2 side. The first covering portion 4 covers plural connecting portions 6 respectively electrically connecting the plural electrodes 232 to the plural electric wires 31. The second covering portion 5 covers the first covering portion 4 and the tip portions 310 of the plural electric wires 31. In the present embodiment, the second covering portion 5 covers a portion of the tip portions 310 of the plural electric wires 31 between the first covering portion 4 and the jacket 33. The second covering portion 5 has a lower elastic modulus than the first covering portion 4. Next, each component of the cable connection structure 1 will be described in detail.

Hereinafter, an axial direction of the cable 3 will be referred to as the axial direction X, one side of the cable 3 in the axial direction X, which is the side where the imaging device 2 is connected thereto, will be referred to as the tip side (i.e., distal end-side) X1, and the opposite side is referred to as the base end side (i.e., proximal end-side) X2.

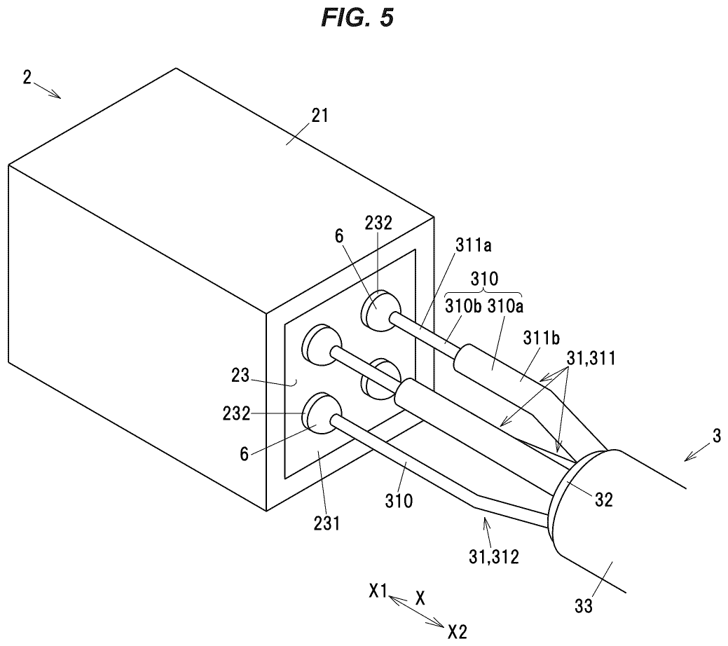

FIG. 5 is a perspective view showing the imaging device 2 and the cable 3. In FIG. 5, the first covering portion (see the reference numeral 4 in, e.g., FIG. 4) and the second covering portion (see the reference numeral 5 in, e.g., FIG. 4) are omitted.

As shown in FIGS. 3 to 5, the imaging device 2 as a whole has a substantially rectangular prism shape that is long in the axial direction X. In the present embodiment, the imaging device 2 is formed so that its outline when viewed in the axial direction X is square. For example, the outline of the imaging device 2 can be a square with each side measuring not less than 0.60 mm and not more than 1.20 mm. In the present embodiment, the field of view of the imaging device 2 is on the tip side X1 in the axial direction X.

As shown in FIG. 3, the imaging device 2 has a rectangular cylindrical body 21 formed along the axial direction X, a light-transmissive imaging window 22 fixed to an end of the cylindrical body 21 on the tip side X1, an imaging element 23 fixed to an end of the cylindrical body 21 on the base end side X2, and plural lenses 24 arranged between the imaging window 22 and the imaging element 23. The imaging element 23 can be, e.g., a CMOS (Complementary Metal Oxide Semiconductor) image sensor, or a CCD (Charge-Coupled Device) image sensor, etc. The imaging device 2 converts information of optical image formed in the imaging element 23 into an electrical signal, and outputs it to the image processor 15 through the cable 3.

As shown in FIG. 5, four electrodes 232 are formed on an electrode formation surface 231 of the imaging element 23 on the base end side X2. The four electrodes 232, when viewed in a direction normal to the electrode formation surface 231, are each formed in a circular shape and are arranged vertically and horizontally in a matrix. In the present embodiment, the four electrodes 232 are arranged at the positions of the vertices of a square. In the present embodiment, the four electrodes 232 consist of a power supply electrode for power supply to imaging device 2, an information output electrode to output an imaging signal converted from the information of the optical image formed in the imaging element 23, a command reception electrode to receive a command signal which is to cause the imaging device 2 to perform a predetermined operation (e.g., an imaging operation, etc.), and a ground electrode connected to a ground potential. Each of the vertical and horizontal pitches of the electrodes 232 can be, e.g., not less than 0.20 mm and not more than 0.50 mm. The four electrodes 232 are respectively electrically connected to the four electric wires 31 of the cable 3.

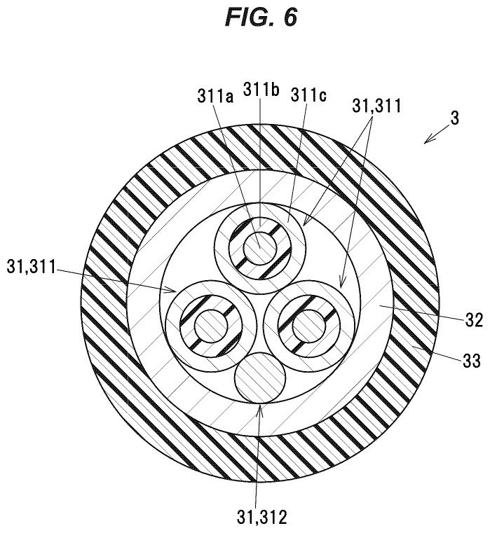

FIG. 6 is a cross-sectional view showing the cable 3. As shown in FIG. 6, the cable 3 is a multicore cable that includes the four electric wires 31, a shield conductor 32 covering all the four electric wires 31, and the jacket 33 covering the shield conductor 32. The four electric wires 31 include three coaxial wires 311 and one drain wire 312. Alternatively, e.g., all the four electric wires 31 may be coaxial wires. The four electric wires 31 may be twisted together or may not be twisted together.

The three coaxial wires 311 consist of a power supply coaxial wire connected to the power supply electrode among the four electrodes 232, an information transmission coaxial wire connected to the information output electrode among the four electrodes 232, and a command transmission coaxial wire connected to the command reception electrode among the four electrodes 232. The power supply coaxial wire supplies electric power to the imaging device 2. The information transmission coaxial wire transmits an imaging signal output from the information output electrode. The command transmission coaxial wire transmits a command signal output from the command reception electrode.

The coaxial wire 311 includes an inner conductor 311a as the core, an inner insulation 311b covering the outer circumference of the inner conductor 311a, and an outer conductor 311c covering the outer circumference of the inner insulation 311b. The inner conductor 311a is a conductor composed of a solid wire or a stranded wire. The conductor is made of, e.g., copper or a copper alloy. The size of the inner conductor 311a can be, e.g., 40 to 50 AWG. AWG stands for American Wire Gauge. The diameter of the inner conductor 311a is selected in consideration of the size of the cable connection structure 1 and the electrical characteristics required of the inner conductor 311a.

The inner insulation 311b surrounding the inner conductor 311a is made of an electrically-insulating resin. The diameter of the inner insulation 311b can be, e.g., not less than 0.10 mm and not more than 0.25 mm.

The outer conductor 311c is formed by spirally winding plural strands so as to be in contact with the outer circumferential surface of the inner insulation 311b. Alternatively, the outer conductor 311c may be formed by braiding plural strands, or can be composed of a metal foil covering the outer circumferential surface of the inner insulation 311b. Furthermore, the outer conductor 311c may be composed of a conductive tape which has a conductive layer on one or both surfaces of a resin strip and is spirally or longitudinally wrapped around the inner insulation 311b, or may be composed of a metal plating film covering the outer circumferential surface of the inner insulation 311b.

The drain wire 312 is connected to the ground electrode. The drain wire 312 is a conductor composed of a solid wire or a stranded wire. The conductor is made of, e.g., copper or a copper alloy. The drain wire 312 is a bare wire with no electrically insulating coating and is at a ground potential by being electrically connected to the outer conductor 311c of each coaxial wire 311 and to the shield conductor 32.

The shield conductor 32 is formed by spirally winding plural strands. The strand is made of, e.g., copper or a copper alloy. Alternatively, the shield conductor 32 may be a braided wire formed by braiding plural strands, or can be composed of a metal foil arranged on the inner circumferential surface of the jacket 33. Furthermore, the shield conductor 32 may be composed of a conductive tape which has a conductive layer on one or both surfaces of an electrically-insulating resin strip and is spirally or longitudinally wrapped around the four electric wires 31. When using a conductive tape having a conductive layer on one surface of an electrically-insulating resin strip, the tape may be wrapped longitudinally or spirally with the conductive layer facing the four electric wires 31 so that the conductive layer serves as the shield conductor 32 and the resin as the jacket 33.

The jacket 33 covers the four electric wires 31 and the shield conductor 32 except for their end portions on the tip side X1. The jacket 33 is made of an electrically insulating resin such as PFA (tetrafluoroethylene-perfluoroalkyl vinyl ether copolymer) or PET (polyethylene terephthalate) and is formed in a cylindrical shape. The shield conductor 32 and the outer conductors 311c of the three coaxial wires 311 are cut off at a position near the end of the jacket 33.

As shown in FIG. 4, in the present embodiment, a portion of the shield conductor 32 on the tip side X1 is exposed from the jacket 33 on the tip side X1 and is covered with the second covering portion 5. In addition, the inner conductors 311a and the inner insulations 311b of the three coaxial wires 311, as well as the drain wire 312, are exposed from the shield conductor 32 and the jacket 33 on the tip side X1. Each of the three coaxial wires 311 and the drain wire 312 has a portion exposed from the shield conductor 32 and the jacket 33 on the tip side X1, and this portion is referred to as the tip portion 310.

The tip portion 310 of the coaxial wire 311 has an insulated portion 310a at which the inner conductor 311a is covered with the inner insulation 311b, and a conductor exposed portion 310b which is located on the tip side X1 relative to the insulated portion 310a and at which the inner conductor 311a is exposed from the inner insulation 311b. The insulated portion 310a is longer than the conductor exposed portion 310b.

The four electric wires 31 are arranged so that their tips respectively face the electrodes 232 as the connection target. In addition, the tip of each of the four electric wires 31 and the electrode 232 as the connection target are mechanically and electrically connected through the connecting portion 6 provided on the electrode 232 as the connection target. That is, the four electric wires 31 and the four electrodes 232 are respectively mechanically and electrically connected through the four connecting portions 6.

The connecting portion 6 is made of a conductive material capable of changing its state between a flowable state and a hardened state due to temperature changes, etc., and is made of, e.g., solder or a conductive resin, etc. The connecting portion 6 is formed in a substantially hemispherical shape that bulges toward the base end side X2.

The imaging device 2, the four connecting portions 6, and a portion of the cable 3 between the electrode formation surface 231 and the jacket 33 are embedded in the first covering portion 4 and the second covering portion 5.

The first covering portion 4 covers all the four connecting portions 6 and reinforces the four connecting portions 6. The first covering portion 4 is made of, e.g., a relatively hard insulating material such as an adhesive of cyanoacrylate, etc. or an epoxy resin. Forming the first covering portion 4 using a relatively hard insulating material makes it easy to reinforce the connecting portions 6.

The first covering portion 4 covers not only the four connecting portions 6 but also an end of a portion of each electric wire 31 exposed from the connecting portion 6 that is the end located on the connecting portion 6 side. The first covering portion 4 does not cover the inner insulations 311b of the electric wires 31 in the present embodiment, but may cover the inner insulations 311b.

In the present embodiment, the first covering portion 4 is formed in a dome shape bulging from the electrode formation surface 231 toward the base end side X2, and has a curved surface portion 41 that bulges so as to be convex toward the base end side X2. This is easily achieved, e.g., by forming the first covering portion 4 and the second covering portion 5 in the order of the first covering portion 4 and the second covering portion 5.

The first covering portion 4 is hard, hence, when its formation range in the axial direction X is too wide, the flexibility of the cable connection structure 1 decreases. Therefore, from the viewpoint of improving the flexibility of the cable connection structure 1, a length L1 of the first covering portion 4 in the axial direction X is preferably short. For example, the length L1 of the first covering portion 4 in the axial direction X is preferably smaller than a diameter D of the jacket 33. As an example, the length L1 of the first covering portion 4 in the axial direction X can be not less than 0.2 mm and less than 0.5 mm.

The second covering portion 5 covers a portion of the cable 3 between the first covering portion 4 and the jacket 33. An end of the second covering portion 5 on the tip side X1 is adhered to the first covering portion 4, and an end of the second covering portion 5 on the base end side X2 is adhered to an end of the jacket 33 on the tip side X1. The second covering portion 5 serves to suppress short circuits respectively between the conductor exposed portions 310b of the coaxial wires 311/the drain wire 312 and other members (e.g., other electric wires 31, conductors arranged around the cable connection structure 1).

The portion of the second covering portion 5 on the tip side X1 is in tight contact with the curved surface portion 41 of the first covering portion 4. That is, a concave curved surface portion 51, which is along the curved surface portion 41 and is recessed toward the base end side X2, is formed on the second covering portion 5 on the tip side X1. In the present embodiment, the end portion of the second covering portion 5 on the base end side X2 also covers the shield conductor 32 exposed from the jacket 33, thereby suppressing short circuits between the shield conductor 32 and other members.

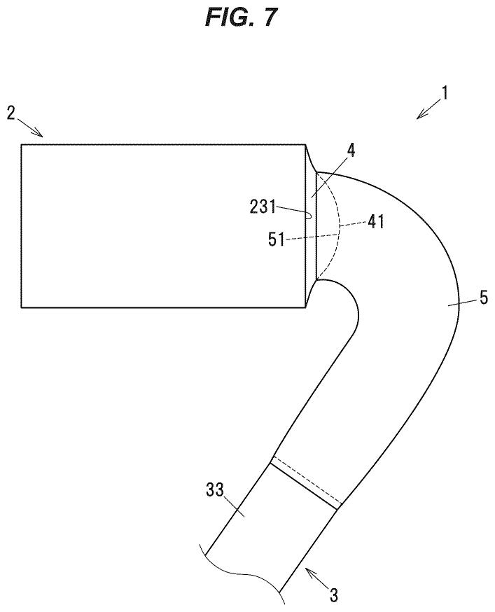

FIG. 7 is a plan view showing an example of a bent state of the cable connection structure 1. The second covering portion 5 is made of a material having a lower elastic modulus than the first covering portion 4. The second covering portion 5 is made of, e.g., an electrically insulating rubber such as silicone rubber. By configuring the second covering portion 5 to have a relatively low elastic modulus, the cable connection structure 1 can be bent at the location where the second covering portion 5 is present, as shown in FIG. 7. This allows the cable connection structure 1 to be bent at a position relatively close to the connecting portions 6, making it easier to mitigate stress concentration on the connecting portions 6. Furthermore, it is possible to reduce the movement of the imaging device 2 when, e.g., changing the viewing direction of the imaging device 2 by using the steering wires described above, making it easier to change the viewing direction even in, e.g., a relatively narrow region inside the subject's body.

For example, the elastic modulus of the second covering portion 5 is preferably less than 1/20 of the elastic modulus of the first covering portion 4. For example, the first covering portion 4 can be formed using a cyanoacrylate-based adhesive material with an elastic modulus of 1700 N/mm², and the second covering portion 5 can be formed using a silicone rubber-based adhesive material with an elastic modulus of 20 N/mm². The elastic modulus of the second covering portion 5 is also preferably lower than the jacket 33.

In addition, as shown in FIG. 4, a length L2 of the second covering portion 5 in the axial direction X is preferably long from the viewpoint of improving the flexibility. For example, the length L2 is preferably longer than the length L1 of the first covering portion 4 in the axial direction X. However, if the length L2 is too long, it will be difficult to form the second covering portion 5, hence, the length L2 is kept to not more than a predetermined length. The length L2 can be, e.g., not less than 1.8 mm and not more than 2.0 mm.

The first covering portion 4 and the second covering portion 5 are formed with a small diameter so as not to stick out from the outline of the imaging device 2 when viewed in the axial direction X.

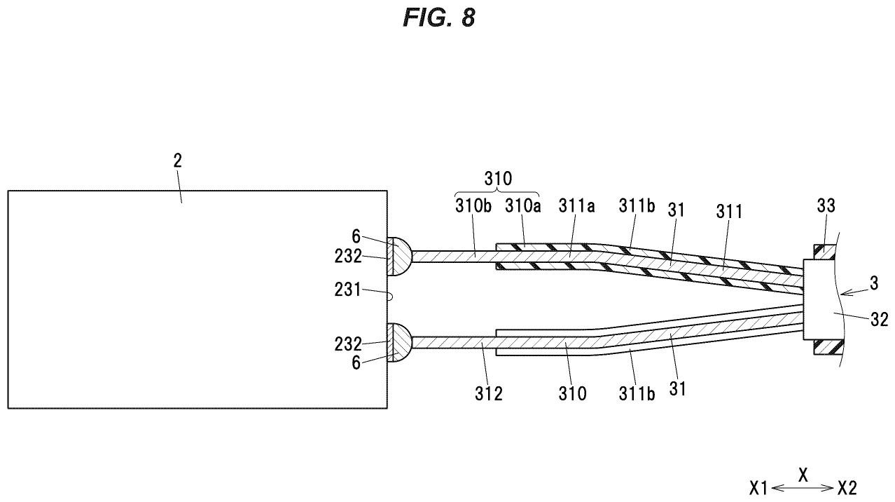

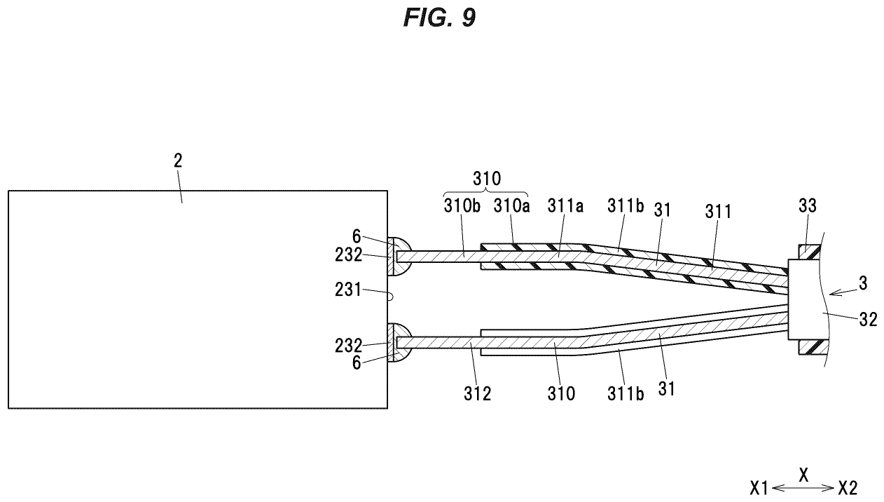

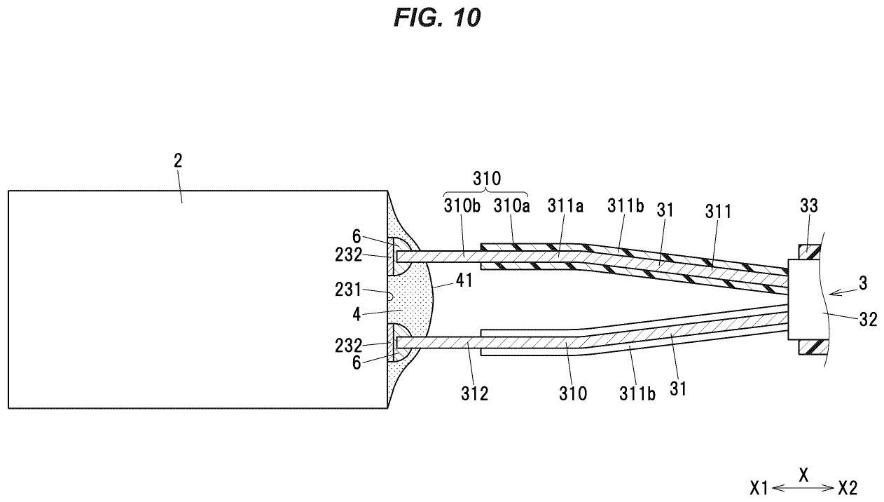

Next, an example of a method for manufacturing the cable connection structure 1 in the present embodiment will be described with reference to FIGS. 8 to 10. FIG. 8 is a cross-sectional view showing the imaging device 2 and the cable 3 in a state before being connected (before soldering). FIG. 9 is a cross-sectional view showing the imaging device 2 and the cable 3 in a connected state (after soldering). FIG. 10 is a cross-sectional view showing a state in which the first covering portion 4 is formed. In FIGS. 8 to 10, the inner insulation 311b depicted around the drain wire 312 is that of the coaxial wire 311 located on the far side of the page relative to the drain wire 312.

First, the imaging device 2 and the cable 3 as described above are prepared. At this time, for the imaging device 2, four connecting portions 6 (i.e., solder) are respectively arranged on the four electrodes 232.

Next, as shown in FIG. 9, the tips of the four electric wires 31 of the cable 3 are butted against the connecting portions 6 provided on the electrodes 232 as the connection target, and are soldered.

Next, as shown in FIG. 10, the first covering portion 4 is formed so as to cover the four connecting portions 6. At this time, the material of the first covering portion 4 which is in a flowable state is applied using a needle-shaped member or a dispenser, etc. so as to cover the four connecting portions 6. As a result, the first covering portion 4 is formed in a dome shape that bulges toward the base end side X2, and the curved surface portion 41 is formed on the surface on the base end side X2. This material is then hardened, thereby obtaining the first covering portion 4. The viscosity of the first covering portion 4 before being hardened is preferably higher than that of the second covering portion 5 before being hardened. Flow of the first covering portion 4 toward the base end side X2 due to capillary action, etc. at the time of applying the first covering portion 4 is thereby suppressed.

Next, a portion from the first covering portion 4 to the tip end of the jacket 33 is covered with the second covering portion 5, as shown in FIG. 4. Also when forming the second covering portion 5, the material of the second covering portion 5 which is in a flowable state is applied using a needle-shaped member or a dispenser, etc. As a result, the second covering portion 5 has the concave curved surface portion 51 which is formed on the end face on the tip side X1 and adhered to the curved surface portion 41 of the first covering portion 4, and a portion of the second covering portion 5 on the base end side X2 covers the end of the jacket 33. This material is then hardened, thereby obtaining the second covering portion 5. As described above, the viscosity of the second covering portion 5 before being hardened is preferably lower than that of the first covering portion 4 before being hardened. As for the second covering portion 5 that is formed longer in the axial direction X than the first covering portion 4 as described above, reducing the viscosity of the material of the second covering portion 5 makes it easier for the material to flow in the axial direction X, thereby facilitating application.

The cable connection structure 1 in the present embodiment can be manufactured as described above.

### Functions and Effects of the Embodiment

The cable connection structure 1 in the present embodiment includes the first covering portion 4 that covers the plural connecting portions 6 respectively electrically connecting the plural electrodes 232 to the plural electric wires 31, and the second covering portion 5 that covers the first covering portion and the tip portions 310 of the plural electric wires 31. Then, the second covering portion 5 has a lower elastic modulus than the first covering portion 4. Therefore, the plural connecting portions 6 are reinforced by the first covering portion 4, and also, the flexibility of the cable connection structure 1 is ensured by the second covering portion 5. Furthermore, in the case where the cable connection structure 1 is assembled to the camera head 13 as in the present embodiment, stress generated in, e.g., the connecting portions 6 during the assembly is reduced since the flexibility of the cable connection structure 1 is ensured.

In addition, in the axial direction X, the length L2 of the second covering portion 5 is longer than the length L1 of the first covering portion 4. Therefore, it is easy to further improve the flexibility of the second covering portion 5.

Furthermore, the length L1 of the first covering portion 4 in the axial direction X of the cable 3 is smaller than the diameter D of the jacket 33. By configuring the length L1 of the relatively hard first covering portion 4 to be short in this manner, it is easy to improve the flexibility of the cable connection structure 1.

In addition, at least one of the plural electric wires 31 (i.e., the drain wire 312) is an uncoated bare wire. In this case, electrical insulation between the bare wire and conductors arranged therearound can be ensured by the first covering portion 4 and the second covering portion 5.

In addition, the contact surfaces of the first covering portion 4 and the second covering portion 5 are curved. In the present embodiment, the first covering portion 4 has the curved surface portion 41 and the second covering portion 5 has the concave curved surface portion 51. Therefore, it is easy to provide a strong contact between the first covering portion 4 and the second covering portion 5.

In addition, the electronic component is the imaging device 2 that includes the imaging element 23 for the endoscope 10. The endoscope 10 is inserted into the body and is also required to have flexibility, and the cable connection structure 1 in the present embodiment is suitably used for such an endoscope 10.

As described above, according to the present embodiment, it is possible to provide a cable connection structure that can reinforce connecting portions respectively between plural electric wires and plural electrodes of an electronic component and also have improved flexibility.

### Modifications

Possible modifications to the embodiment will be described.

For example, the imaging device 2 has a field of view on the tip side X1 in the axial direction X of the cable 3, but it is not limited thereto. The field of view may be, e.g., in a direction intersecting the axial direction X of cable 3.

In addition, the example in which the cable 3 has the four electric wires 31 connected to the imaging device 2 has been described, but the configuration is not limited thereto and, e.g., electric wires connected to components other than imaging device 2 may be included. An electric wire connected to an LED, etc., may be arranged in the cable 3, as an example.

In addition, each of the first covering portion 4 and the second covering portion 5 is a one-piece component, but it is not limited thereto. For example, the second covering portion 5 may be composed of plural rubber members having a lower elastic modulus than the first covering portion 4.

### Summary of the Embodiment

Next, technical ideas understood from the embodiment will be described below citing the reference signs, etc. used for the embodiment. However, each reference sign, etc. described below is not intended to limit the constituent elements in the claims to the members, etc., specifically described in the embodiment.

According to the first feature, a cable connection structure 1 comprises: an electronic component 2 comprising a plurality of electrodes 232; a cable 3 comprising a plurality of electric wires 31, and a jacket 33 that covers the plurality of electric wires 31 while exposing tip portions 310 of the plurality of electric wires 31 on the electronic component 2 side; a first covering portion 4 that covers a plurality of connecting portions 6 respectively electrically connecting the plurality of electrodes 232 to the plurality of electric wires 31; and a second covering portion 5 that covers the first covering portion 4 and the tip portions 310, wherein the second covering portion 5 has a lower elastic modulus than the first covering portion 4.

According to the second feature, in the cable connection structure 1 as described in the first feature, in an axial direction X of the cable 3, a length L2 of the second covering portion 5 is longer than a length L1 of the first covering portion 4.

According to the third feature, in the cable connection structure 1 as described in the first or second feature, the length L1 of the first covering portion 4 in the axial direction X of the cable 3 is smaller than a diameter D of the jacket 33.

According to the fourth feature, in the cable connection structure 1 as described in any one of the first to third features, at least one of the plurality of electric wires 31 comprises an uncoated bare wire.

According to the fifth feature, in the cable connection structure 1 as described in any one of the first to fourth features, contact surfaces of the first covering portion 4 and the second covering portion 5 are curved.

According to the sixth feature, in the cable connection structure 1 as described in any one of the first to fifth features, the electronic component 2 comprises an imaging device 2 comprising an imaging element 23 for an endoscope 10.

### Additional Note

Although the embodiment of the invention has been described, the invention according to claims is not to be limited to the embodiment described above. Further, please note that not all combinations of the features described in the embodiment are necessary to solve the problem of the invention. In addition, the invention can be appropriately modified and implemented without departing from the gist of the invention.

1. A cable connection structure, comprising:
an electronic component comprising a plurality of electrodes;
a cable comprising a plurality of electric wires, and a jacket that covers the plurality of electric wires while exposing tip portions of the plurality of electric wires on an electronic component-side;
a first covering portion that covers a plurality of connecting portions respectively electrically connecting the plurality of electrodes to the plurality of electric wires; and
a second covering portion that covers the first covering portion and the tip portions, wherein the second covering portion has a lower elastic modulus than the first covering portion.

2. The cable connection structure according to claim 1, wherein in an axial direction of the cable, a length of the second covering portion is longer than a length of the first covering portion.

3. The cable connection structure according to claim 1, wherein a length of the first covering portion in an axial direction of the cable is smaller than a diameter of the jacket.

4. The cable connection structure according to claim 2, wherein the length of the first covering portion in the axial direction of the cable is smaller than a diameter of the jacket.

5. The cable connection structure according to claim 1, wherein at least one of the plurality of electric wires comprises an uncoated bare wire.

6. The cable connection structure according to claim 2, wherein at least one of the plurality of electric wires comprises an uncoated bare wire.

7. The cable connection structure according to claim 1, wherein contact surfaces of the first covering portion and the second covering portion are curved.

8. The cable connection structure according to claim 2, wherein contact surfaces of the first covering portion and the second covering portion are curved.

9. The cable connection structure according to claim 1, wherein the electronic component comprises an imaging device comprising an imaging element for an endoscope.

10. The cable connection structure according to claim 2, wherein the electronic component comprises an imaging device comprising an imaging element for an endoscope.
